(19) United States
(12) Patent Application Publication
(10) Pub. No.: US 2025/0201413 A1
(43) Pub. Date: Jun. 19, 2025

(54) PREDICTING HEALTH-RELATED EVENTS USING NEURAL NETWORKS

(71) Applicant: Google LLC, Mountain View, CA (US)

(72) Inventors: Gregory Sean Corrado, San Francisco, CA (US); Michael Dwight Howell, Palo Alto, CA (US)

(21) Appl. No.: 18/982,456

(22) Filed: Dec. 16, 2024

Related U.S. Application Data
(60) Provisional application No. 63/610,371, filed on Dec. 14, 2023.

(51) Int. Cl. G16H 50/20; G06N 3/0475; G16H 50/50

(52) U.S. Cl. CPC G16H 50/20; G06N 3/0475; G16H 50/50

(57) ABSTRACT

Methods, systems, and apparatus, including computer programs encoded on a computer storage medium, for generating a prediction of a health-related event. One of the methods includes: identifying health-related data associated with an individual, the health-related data comprising a sequence of health-related events; generating, for each health-related event in the sequence of health-related events, a respective embedded representation of the health-related event; generating, conditioned on at least the respective embedded representations of the health-related events, an output embedded representation of the future health-related event; and processing at least the output embedded representation of the future health-related event to generate the prediction of the future health-related event.

### CROSS-REFERENCE TO RELATED APPLICATION

This application claims priority to U.S. Provisional Application No. 63/610,371, filed on Dec. 14, 2023. The disclosure of the prior application is considered part of and is incorporated by reference in the disclosure of this application.

### BACKGROUND

This specification relates to processing sequences of health events using neural Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

### SUMMARY

This specification describes a system implemented as computer programs on one or more computers in one or more locations that generates a prediction of a health-related event using one or more neural networks.

In general, the system generates a prediction of a health-related event associated with an individual, conditioned on health-related data associated with the individual. The health-related data can include data identifying health related-events at multiple points of time. For example, the health-related data can include a sequence of health-related events. The health-related events in the sequence can be ordered by time, so that the most-recently occurring health-related event is the last health-related event in the sequence.

The system can be configured to perform any of a variety of tasks that require generating a prediction of a health-related event, also referred to as a health event. For example, the system can generate predictions of future health-related events, or missing health-related events.

Particular embodiments of the subject matter described in this specification can be implemented so as to realize one or more of the following advantages. A neural network can effectively be used to analyze a sequence of health events, e.g., a sequence of health events derived from an electronic medical record for a current patient. For example, the system described in this specification can generate one or more predicted future health-related events for a sequence of health events by generating a respective embedded representation of each health-related event. The system can generate an output embedded representation of a predicted future health-related event using a sequence processing neural network. The system can generate the predicted future health-related event using one or more de-embedding machine learning models. As another example, the system described in this specification can identify missing health-related events in the sequence of health events. For example, the system can generate a sequence of predicted future health-related events. The system can receive updated health-related data. The system can identify health-related events in the sequence of predicted future health-related events as missing health-related events if equivalents do not occur in the updated health-related data.

A doctor or other healthcare professional can be provided with information characterizing the output of the neural network or outputs derived from outputs generated by the neural network, improving the healthcare professional's ability to provide quality healthcare to the professional's patients or to perform risk assessment. For example, the system can simulate the forward evolution of a medical case associated with a patient or in aggregate. For example, the system can simulate the forward evolution of a medical case associated with a patient by processing health-related data for the patient to generate one or more predicted future health-related events. The system can simulate the forward evolution of multiple patients in aggregate by processing health-related data for the multiple patients to generate one or more predicted future health-related events for each of the multiple patients.

As an example, a healthcare professional can be provided with useful information about future health events that may become associated with a current patient, e.g., health events that are likely to be the next health event to be associated with the patient. For example, the system described in this specification can process health-related data for the patient to generate predictions of future health events. For example, the system can use a sequence processing neural network that is configured to generate an output embedded representation of a predicted future health-related event conditioned on a sequence of health-related events.

In some examples, the future health events can include actions. The system can suggest, evaluate the impact of, and score the actions for the patient. The system can provide this information to the healthcare professional. For example, in some cases, a predicted future health event can be an action such as a prescription. The system can provide data representing the action for display to the healthcare professional. The healthcare professional can consider the action as a suggestion. As another example, the system can evaluate the impact of the action without having to perform the action. For example, the system can process the health-related data and the action to generate a predicted future health-related event that is a test result. The system can provide data representing the test result to the healthcare professional. The healthcare professional can consider the predicted impact of taking the action without having to take the action. As another example, the system can score the action. For example, the system can determine the score for the action based on a likelihood and/or the impact of taking the action. The system can provide data representing the score for display to the healthcare professional. The healthcare professional can consider the score as guidance for whether to take the action.

In some examples, the system can provide a user interface that allows a healthcare professional to provide a future window of time, and the system can provide predictions of future health events likely to occur within the window of time. For example, the system can process health-related data and any previously predicted future health-related events for the patient to generate one or more predicted future health-related events until one of the predicted future health-related events occurs after the window of time. Generating a prediction of a future health-related event can also aid in administrative reasoning, for healthcare quality and patient safety monitoring, or in population management. For example, the system can be used for healthcare monitoring to identify unlikely proposed health-related events in the context of the health data, such as commission events or suboptimal decisions. As another example, the system can be used for healthcare monitoring to identify missing events that are likely to have occurred but did not occur.

Additionally, the healthcare professional can be provided with information that identifies the potential effect of a proposed treatment on the patient's health. For example, the system can provide a user interface that allows a healthcare professional to provide proposed health events for a sequence of health events. The system can process the sequence of health events including the proposed health event and provide a predicted future health event. The predicted future health event can represent the impact of the proposed health event. The system can thus allow a healthcare professional to simulate what may happen given their proposed actions and be used as a decision aid for healthcare professionals considering different courses of clinical actions. The system can also aid in the training and evaluation of healthcare professionals. Additionally, in some cases, an alert can be generated for a healthcare professional that is triggered if an action the healthcare professional proposes to take causes a negative impact to the health of the patient.

Furthermore, an alert can be generated for a healthcare professional that is triggered if an action the healthcare professional proposes to take is unusual in the context of the sequence of health events, also referred to as a commission event or a suboptimal decision. For example, the system can receive data indicating a proposed action to take. The system can process a distribution over future health-related events to determine a likelihood for the action the healthcare professional proposes to take. The system can determine that the likelihood for the action the healthcare professional proposes to take is below a threshold likelihood, and in response, generate an alert that the proposed action is unusual or is an outlier, reducing the likelihood of negative impacts on a patient's health due to commission events or suboptimal decisions.

Missing events can be difficult to infer or detect because practice patterns can vary, and healthcare systems often do not have fixed or consistent sampling intervals. The system can be used to infer missing events in a patient's medical record. For example, the system can identify a serialized medical history missing some health events, and reason about the missing health events. Missing events can include an omission event, which can include an action that was not proposed or taken but is predicted to be high probability. For example, the system can determine that a particular action, such as a thyroid stimulating hormone (TSH) blood test, has a high likelihood of having occurred in the medical history, but was not in the medical history. As another example, the system can determine that a potassium medication order is likely after a low blood potassium level result from a lab test, but the potassium medication order was not in the medical history. The system can thus prompt the healthcare professional to consider taking the particular action, reducing the likelihood of negative impacts on a patient's health due to omission events.

Additionally, the system can be used as part of a larger medical intelligence system, to codify standard medical practice, to discover patterns in treatment and outcomes, to analyze existing medical techniques or healthcare systems, or to make novel recommendations or facilitate scientific discoveries. For example, the system can allow for the identification of missing health events in health-related data, enabling analysis of existing healthcare systems. As another example, the system can generate predictions of future health-related events for health-related data for multiple patients, enabling the discovery of patterns in treatment and outcomes across multiple patients.

The system can provide for data security and data privacy of health-related data. For example, the system can identify health-related data and generate embedded representations of the health-related events of the health-related data on one computer. For example, the system can generate the embedded representations on the user device. The system can provide the embedded representations to the sequence processing neural network, which may be executing on one or more computers remote from the user device, rather than directly providing the health-related data to one or more computers remote from the user device. As another example, in implementations where the system receives embedded representations of health-related events and generates an output embedded representation of the prediction of a future health-related event using a sequence processing neural network, the system that executes the sequence processing neural network may not have access to the health-related data.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example health-related event prediction system.

FIG. 2 shows an example process for generating a prediction of a future health-related event.

FIG. 3 is a flow chart of an example process for generating a predicted future health-related event associated with an individual.

FIG. 4 is a flow chart of an example process for generating an embedded representation of a health-related event.

FIG. 5 is a flowchart of an example process for processing an output embedded representation using a de-embedding machine learning model.

FIG. 6 is a flowchart of another example process for processing an output embedded representation using a de-embedding machine learning model.

FIG. 7 is a flowchart of an example process for obtaining a prediction of a future health-related event associated with an individual.

FIG. 8 is a flowchart of an example process for obtaining a prediction of a future health-related event associated with an individual.

FIG. 9 is a flowchart of an example process for generating a prediction of a future health-related event associated with an individual.

FIG. 10 shows an example health-related event prediction system.

FIG. 11 is a flowchart of an example process for generating a prediction of a missing health-related event associated with an individual.

FIG. 12 shows an example health-related event detection system.

FIG. 13 is a flowchart of an example process identifying one or more missing health-related events associated with an individual.

Like reference numbers and designations in the various drawings indicate like elements.

### DETAILED DESCRIPTION

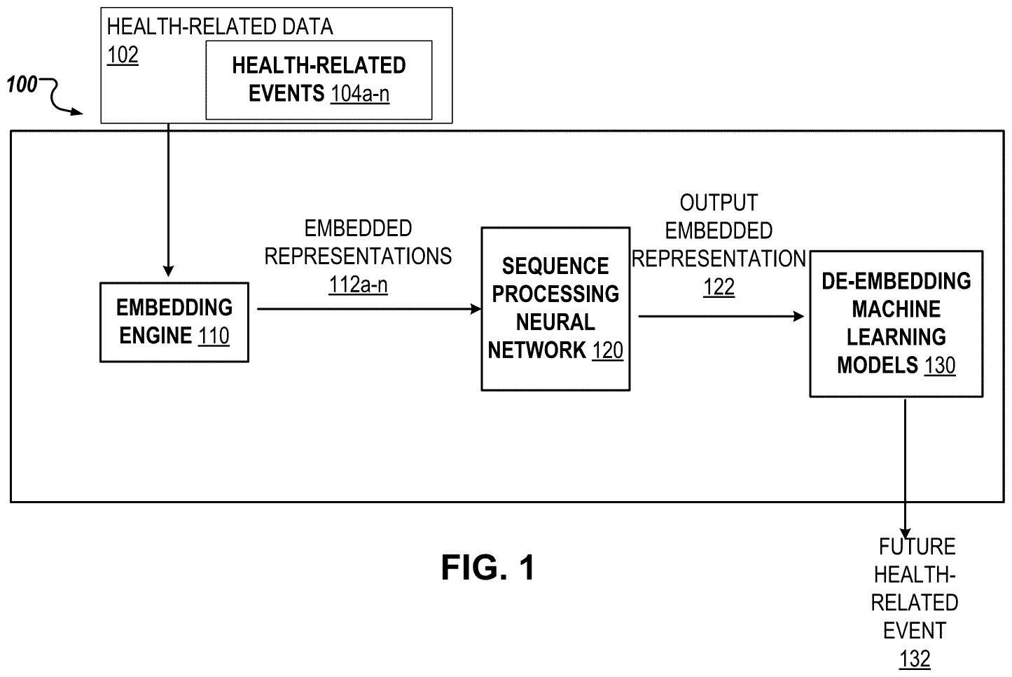

FIG. 1 shows an example health-related event prediction system 100. The health-related event prediction system 100 is an example of a system implemented as computer programs on one or more computers in one or more locations in which the systems, components, and techniques described below are implemented.

The health-related event prediction system 100 is configured to process health-related data 102 associated with an individual to generate a predicted future health-related event 132 for the individual. Each health-related event of the health-related 104a-n can be associated with an event type that is one of multiple possible event types. The predicted future health-related event 132 is associated with an event type that is one of the multiple possible event types.

To generate a prediction of a future health-related event associated with the individual, the system identifies health-related data associated with the individual. The health-related data 102 can include data identifying health related-events 104a-n at multiple points of time. For example, the health-related data 102 can include a sequence of health-related events 104a-n.

Each health-related event of the health-related 104a-n and the future health-related event 132 can be associated with an event type. Example event types include symptoms, tests (also referred to as labs), test results (also referred to as lab results), diagnoses, medications, prescriptions, referrals, actions (e.g., procedures), outcomes, patient information records (e.g., age, height, weight) and so on. The event types may also include health-related images, e.g., X-Ray or other diagnostic images, health-related electronic documents, e.g., free-form notes generated by a doctor during an appointment, or Subjective, Objective, Assessment, and Plan (SOAP) notes, or Digital Imaging and Communications in Medicine (DICOM) images. As an example, this specification will refer to an individual that is a patient, although the system can also generate predictions of health-related events for individuals that are not patients.

The event types can also include other data that may be classified as impacting the health of the patient, such as genomic data or sensor data. For example, the event types can include data characterizing a patient's activity or other health-related data collected by a patient's devices, e.g., activity tracking devices or activity tracking applications executing on mobile devices. For example, the activity data can include data identifying distances travelled by a patient on a particular day, workout or other fitness activity engaged in by the patient, meals eaten by the patient, and so on. The event types can also include other data that may be considered to impact the health of the patient, e.g., prescription fulfillment data for the patient or data identifying time-varying air quality measures over a time period of interest.

The health-related data 102 can also include time information for each health-related event. For example, the health-related data can include data identifying the time each health-related event 104a-n occurred, e.g., a timestamp for the health-related event, or a relative time each health-related event 104a-n occurred relative to other events in the sequence, e.g., an amount of time elapsed since a previous health-related event occurred.

In some examples, each health-related event can be represented as a Fast Healthcare Interoperability Resources (FHIR) resource. Each FHIR resource defines one or more features of the health event, e.g., event type, value or content, or time information.

The system can generate a respective embedded representation 112a-n of each health-related event 104a-n in the sequence of health-related events 104a-n. For example, the system 100 can process each of the health-related events 104a-n using an embedding engine 110 to generate the respective embedded representations 112a-n. Each of the respective embedded representations 112a-n can include one or more embeddings in a shared embedding space that is shared across the possible event types.

Embedded representations for clinically similar health-related events are located close to each other in the shared embedding space. For example, prescriptions for different doses of medications that have similar effects may be located close to each other. As another example, prescriptions for medications of the same medication family may be located close to each other. As another example, medical tests that measure the same types of information may be located close to each other. As another example, medications that are paired with lab tests, such as lab tests given before certain medications are prescribed, or monitoring tests given while certain medications are being taken, may be located close to each other.

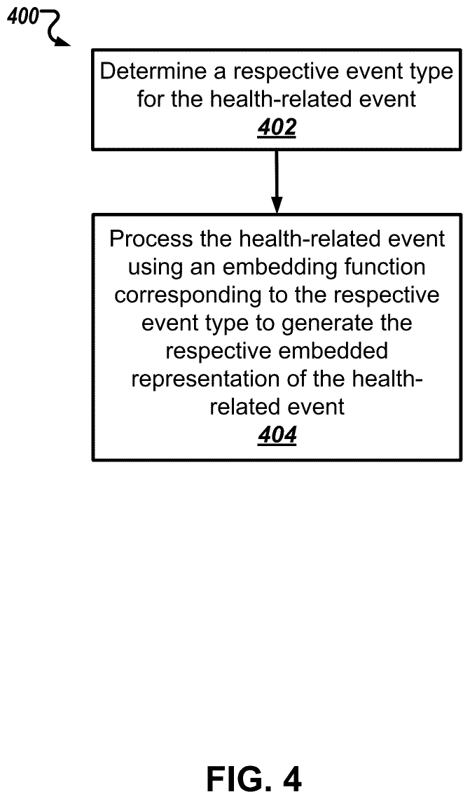

In some examples, embedded representations for health-related events of the same event type are located closer to each other in the shared embedding space. For example, as described below with reference to FIG. 4, the system can apply an event type encoding or a learned transformation for the event type to the embedded representation of each health-related event.

In some implementations, embedded representations for health-related events of different event types can include different numbers of embeddings in the shared embedding space. For example, the embedded representation for an MRI image may include multiple vectors, and the embedded representation for a prescription or a medication dose may include one vector.

To generate an embedded representation of each health-related event in the sequence, the system 100 can use normalization and embedding functions. Each event type can have its own normalization function and embedding function. In some examples, the system 100 can normalize the embeddings of the embedded representations generated by the embedding functions.

The embedding engine 110 is configured to generate a respective embedded representation for a given health-related event. In some examples, the embedding engine 110 includes multiple embedding functions. Each embedding function can correspond to a particular type of health-related event and can map health-related events of the particular type to embedded representations of one or more embeddings in a shared embedding space. For example, for each health-related event in the sequence, the system can determine an event type for the health-related event, and process the health-related event using an embedding function corresponding to the event type to generate the embedded representation for the health-related event.

Embedding functions can be designed, constructed, or learned. The embedding functions can include classifiers, probabilistic models, language models, etc. For example, one or more of the embedding functions can include embedding neural networks.

Example embedding functions and generating a respective embedded representation for a health-related event associated with an event type are described below in further detail with reference to FIG. 4.

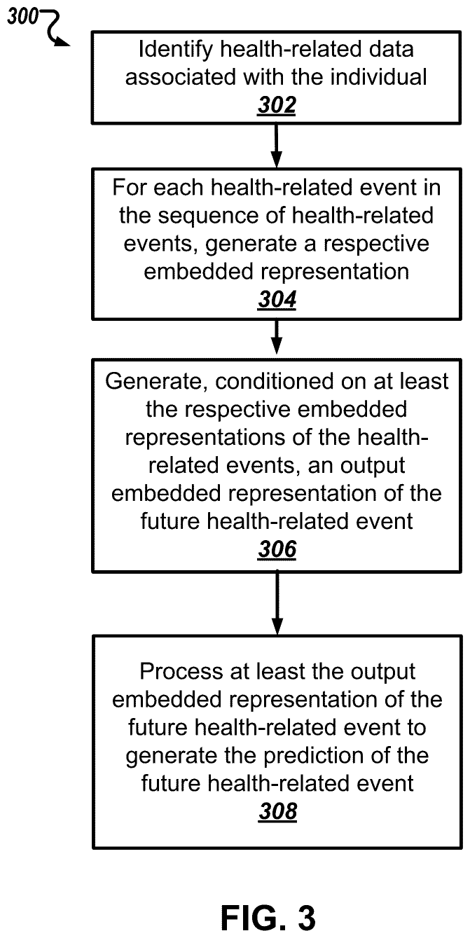

The system can use a sequence processing neural network 120 to generate an output embedded representation 122 of the future health-related event 132 conditioned on at least the embedded representations 112a-n of the health-related events 104a-n in the sequence. The sequence processing neural network 120 can convert the sequence of embedded representations 112-an into the output embedded representation 122 that represents a next predicted health-related event. The sequence processing neural network 120 can include a neural network such as a Transformer network, a recurrent neural network (RNN), a Long Short-Term Memory (LSTM) network, or a sequence to sequence (seq2seq) network. The sequence processing neural network is described in further detail below with reference to FIG. 3.

The system can process at least the output embedded representation 122 to generate the prediction of the future health-related event 132. For example, the system can process at least the output embedded representation 122 using one or more de-embedding machine learning models 130 to generate the prediction of the future health-related event 132. In some examples, the prediction of the future health-related event 132 can include an FHIR resource for the future health-related event 132.

The de-embedding machine learning models 130 can include, e.g., classifiers, probabilistic models, neural networks, language models, etc. In some implementations, each of the one or more de-embedding machine learning models 130 can correspond to a particular event type. The system 100 can process the output embedded representation 122 using the de-embedding machine learning model that corresponds to a particular event type, e.g., obtained from a user or determined by the system 100, to generate the prediction of the future health-related event. Generating the prediction of the future health-related event is described in more detail with reference to FIG. 3.

The system 100 can generate predictions of multiple future health-related events associated with the patient. For example, the system 100 can generate a sequence of multiple future health-related events by generating each future health-related event in the sequence conditioned on a current input sequence of health-related events that includes the health-related events 104a-n and the health-related events that have already been generated by the system. For example, the system 100 can generate a first prediction of a future health-related event 132 given a sequence of health-related events 104a-n, and update the sequence of health-related events 104a-n to include the first prediction 132 in the sequence of health-related events. The system can then generate a second prediction of a future health-related event given the updated sequence of health-related events.

As another example, the system 100 can generate each future health-related event in the sequence conditioned on a current input sequence of embedded representations that includes the embedded representations 112a-n and any previously generated output embedded representations.

In some implementations, the system 100 can provide the predictions of the future health-related event 132, or data derived from the prediction of the future health-related event 132, for presentation to a user through a user interface on a user device. In some examples, the user interface can include a graphical user interface. In some examples, the system can provide the predictions of the future health-related event 132, or data derived from the prediction of the future health-related event 132, through audio, e.g., using a text-to-speech system.

In some examples, the system 100 can process the predicted future health-related events to generate a natural language description of the predicted future health-related events. In some examples, the system 100 can generate the natural language description in response to receiving a query from the user for a natural language description. For example, the system 100 can provide data representing the predicted future health-related events and an instruction to generate a natural language description as input to a language model neural network to generate the natural language description. As another example, the system 100 can process one or more features of the FHIR resources for the predicted future health-related events to generate the natural language description. The system 100 can provide the natural language description of the predicted future health-related events to the user through the user interface.

In some examples, the system 100 can process the predicted future health-related events to generate an explanation of the predicted future health-related events. In some examples, the system 100 can generate the explanation in response to receiving a query from the user for an explanation. For example, the system 100 can provide data representing the predicted future health-related events and data representing the health-related data 102, and an instruction to generate an explanation for the predicted future health-related events, as input to a language model neural network to generate the explanation. The system 100 can provide the explanation to the user through the user interface. In some examples, the explanation can include a likelihood of the predicted future health-related events. For example, an explanation can include “since the patient has just initiated warfarin, it is >95% likely that a PT/INR will be ordered.”

In some examples, the system 100 can process the predicted future health-related events and a query about the predicted future health-related events to generate an answer responsive to the query. In some examples, the system 100 can receive the query as a text input through the user interface. In some examples, the system 100 can receive the query as an audio input and convert the audio input to text, e.g., using a speech-to-text system.

For example, the query from the user may include “if I consulted cardiology, what is their consult note likely to say? Just the assessment and plan.” The system can process the health-related data for the patient and determine a predicted future health-related event that has an event type of a consult note as described above. The system 100 can process at least the predicted health-related event to generate the answer. For example, the system 100 can provide the query, data representing the health-related data 102 and the predicted health-related event as input to a language model neural network to generate the assessment and plan. The system can provide the assessment and plan to the user through the user interface.

As an example, the system 100 can provide the answer responsive to the query:

### Assessment:

68-year-old with type 2 myocardial infarction. No evidence of STEMI, so elevated troponin almost certainly related to underlying septic shock from pneumococcal pneumonia.
Plan:
   No need for cardiac catheterization
   Continue home aspirin and statin.
   Hold beta blocker and ARB while in shock
   transthoracic echo to assess LVEF and ensure no obvious seeding of valves from pneumococcal bacteremia

As another example, the system 100 can provide for a conversational user experience. For example, the system 100 can process the health-related data 102, any predicted future health-related events, any proposed predicted future health-related events, or all of the above, to generate answers to queries from a user. As an example, the health-related data 102 can include health-related events for a 32-year-old woman with asthma complicated by respiratory failure, on a ventilator with settings of X, Y, and Z. The query from the user may include “What would happen if I decreased Z by 20%, and also stopped ABX? Play it forward four hours.”

The system 100 can process the health-related data 102 for the patient and determine one or more predicted future health-related events. For example, the system 100 can generate predicted future health-related events that are predicted to occur in the next four hours after the latest health-related event in health-related data 102. The predicted future health-related events can include lab results or measures that indicate a worsened respiratory status. The system 100 can provide data representing the predicted future health-related events for the next four hours for presentation to the user.

The system 100 can receive a further query from the user. For example, the query may include “OK, let's put Z back up by 20%, and try increasing Y by 30%. Play it forward four hours.” The system can process the health-related data 102 for the patient and determine one or more predicted future health-related events. The predicted future health-related events can include lab results or measures that indicate an improved respiratory status. The system 100 can provide data representing the predicted future health-related events for the next four hours for presentation to the user.

The system 100 can receive a further query from the user. For example, the query may include “OK, that seems better. What's the most likely time we'll get to extubation?” The system can process the health-related data and any previously predicted future health-related events for the patient to generate one or more predicted future health-related events that include an action that is an extubation. For example, the system can continuously generate predicted future health-related events until the system determines that a predicted future health-related event is an action that is an extubation. The system can determine time information, e.g., absolute or relative time information, from the predicted future health-related event. For example, in cases where the predicted future health-related event is represented as an FHIR resource, the system can extract the time information from the FHIR resource. The system 100 can provide data representing the relative time or absolute time of the extubation for presentation to the user. For example, the system 100 can provide the answer “The most likely estimate is 96 hours” for presentation to the user.

In some examples, the system 100 can determine a probability that a predicted future health-related event is an extubation. For example, the system 100 can compute the likelihood of the additional predicted future health-related event that is an extubation based on the logits (e.g., scores or inputs to a soft-max layer) output by the sequence processing neural network. The system 100 can provide data representing the likelihood of the extubation for presentation to the user.

In some examples, the system 100 can compute statistics for the predicted health-related events and provide the computed statistics for presentation to the user. For example, the system 100 can generate multiple sequences of predicted health-related events for the health-related data 102. The system 100 can determine the proportion of the sequences of predicted health-related events that include a particular health-related event at particular times. The system 100 can provide data representing the proportion to the user, e.g., in the form “The most likely estimate for time to extubation is 96 hours, but in 25% of cases it is in 5 hours.”

In some implementations, the embedding functions of the embedding engine 110, the sequence processing neural network 120, and/or de-embedding machine learning models 130 can execute separately from a user device. For example, in some implementations, the embedding functions can execute on the user device, and the sequence processing neural network 120 and de-embedding machine learning models 130 can run on one or more computers independently from the user device.

In some implementations, components of the system 100 that include the embedding functions of the embedding engine 110, the sequence processing neural network 120, and/or de-embedding machine learning models 130 can execute separately from each other. For example, the embedding functions can execute on one or more computers, and the sequence processing neural network 120 and/or de-embedding machine learning models 130 can execute on one or more computers remote from the computers that execute the embedding functions. As another example, the sequence processing neural network 120 can execute on one or more computers, and the embedding functions and/or de-embedding machine learning models 130 can execute on one or more computers remote from the computers that execute the sequence processing neural network 120. As another example, the de-embedding machine learning models 130 can execute on one or more computers, and the embedding functions and/or sequence processing neural network 120 can execute on one or more computers remote from the computers that execute the de-embedding machine learning models 130.

In some implementations, the components can communicate with each other over a network. In some implementations, the components can communicate using one or more application programming interfaces (APIs). For example, the system 100 can use an API for the sequence processing neural network 120 to provide inputs and receive outputs to the sequence processing neural network 120.

In some implementations, the system 100 can include any combination of embedding functions of the embedding engine 110, the sequence processing neural network 120, and de-embedding machine learning models 130.

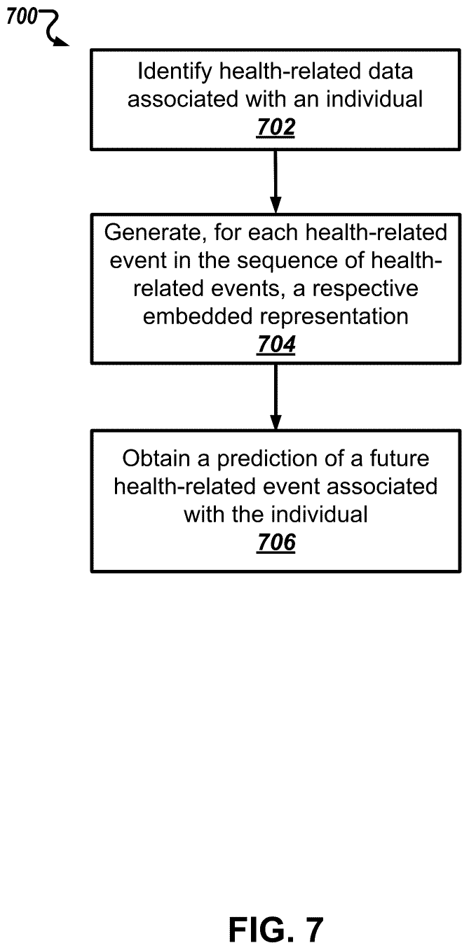

For example, the system can include embedding functions. The system can provide the embedded representations generated using the embedding functions as input to one or more machine learning models, and receive a prediction of a future health-related event. The one or more machine learning models can be part of a separate system. Obtaining a prediction of a future health-related event using a system that includes embedding functions is described below with reference to FIG. 7.

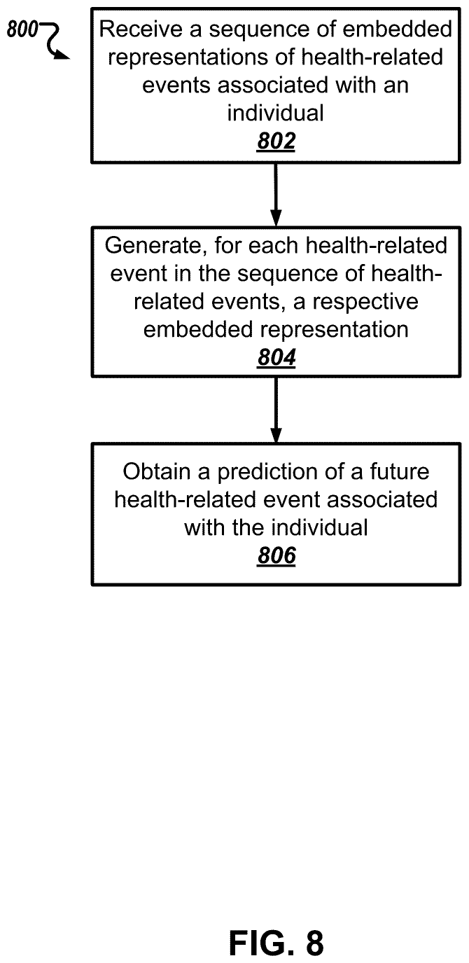

As another example, the system can include a sequence processing neural network. The system can receive embedded representations, process the embedded representations using the sequence processing neural network, and provide an output embedded representation generated by the sequence processing neural network to one or more machine learning models to generate the prediction of the future health-related event. The system can receive embedded representations from a separate system, and provide the output embedded representation to another system. Obtaining a prediction of a future health-related event using a system that includes a sequence processing neural network is described below with reference to FIG. 8.

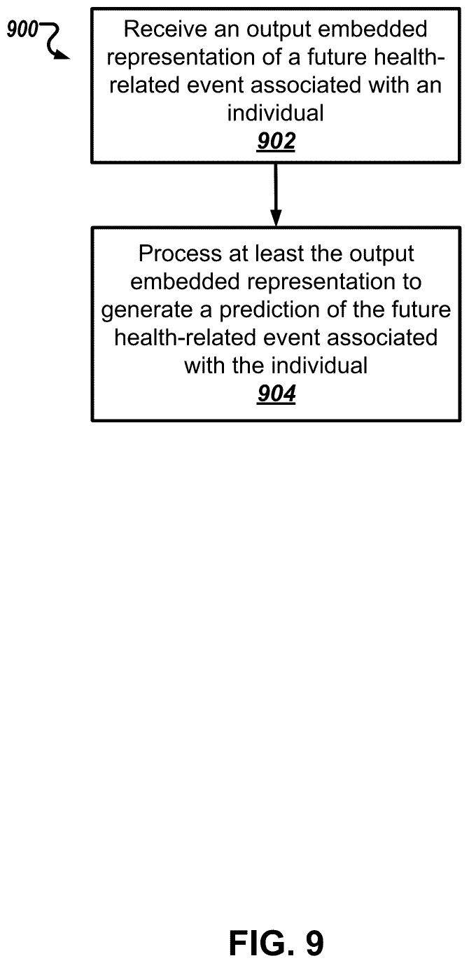

As another example, the system can include one or more de-embedding machine learning models. The system can receive an output embedded representation, and generate the prediction of the future health-related event using the one or more de-embedding machine learning models. The system can receive the output embedded representation from another system. Obtaining a prediction of a future health-related event using a system that includes one or more de-embedding machine learning models is described below with reference to FIG. 9.

As another example, the system can include the embedding functions and the de-embedding machine learning models. The system can provide the embedding representations to another system, and receive an output embedded representation from the other system. The system can generate the prediction of the future health-related event using the de-embedding machine learning models.

Running the sequence processing neural network 120 on a separate system than the embedding functions and de-embedding machine learning models 130 can provide for data security and data privacy of health-related data. For example, the health-related data does not need to be transmitted to the other system. Rather, embedded representations of the health-related data are transmitted to the other system. In addition, the sequence processing neural network generates an output embedded representation of the prediction of the future health-related event. The output embedded representation is transmitted to the system, also providing for the security of the predicted future health-related event.

In some examples, one or more of the health-related events 104a-n are recorded or actual health-related events, e.g., associated with the individual. In these examples, the recorded or actual health-related events can be de-identified. For example, the de-identified recorded or actual health-related events do not include identifying information about names, addresses, contact information, identifiers, etc., for the individual or healthcare professionals. In some examples, the system processes recorded or actual health-related events to generate de-identified recorded or actual health-related events. For example, for each health-related event, the system can remove any identifying information from the health-related event to generate the de-identified health-related event.

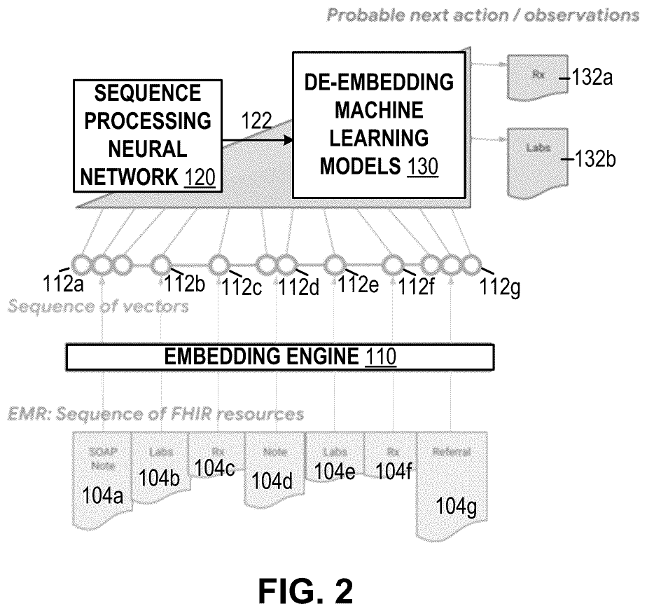

FIG. 2 shows an example process 200 for generating a prediction of a future health-related event. For convenience, the process 200 will be described as being performed by a system of one or more computers located in one or more locations. For example, a health-related event prediction system, e.g., the health-related event prediction system 100 depicted in FIG. 1, appropriately programmed in accordance with this specification, can perform the process 200.

The system identifies health-related data 102. In the example of FIG. 2, the health-related data 102 includes a sequence of health-related events represented as FHIR resources. In particular, the sequence of FHIR resources includes a SOAP Note 104a, a test result (“labs”) 104b, a prescription (“Rx”) 104c, a freeform note (“Note”) 104d, a test result (“labs”) 104e, a prescription (“Rx”) 104f, and a referral 104g.

The system generates a respective embedded representation of each health-related event. Each embedded representation 112a-g includes one or more embeddings in an embedding space. In the example of FIG. 2, embedded representations for health-related events of different event types include different numbers of embeddings in the shared embedding space. For example, the embedded representation 112a for the SOAP Note 102a includes three embeddings, and the embedded representation 112c for the prescription 104c includes one embedding.

The system can generate an output embedded representation 122 of the future health-related event 132a by processing the sequence of embedded representations 112a-g for the health-related events using the sequence processing neural network 120. The embedded representations 112a-g can be ordered within the sequence according to a time at which the corresponding health-related event occurred.

The system processes the output embedded representation 122 using the one or more de-embedding machine learning models 130 to generate the future health-related event 132. For example, the system can process the output embedded representation 122 using a de-embedding machine learning model associated with an event type of prescription. The system 100 generates the health-related event (“Rx”) 132a that has the event type of prescription. In this example, the prescription is an action. The system can provide data representing the action for display to a user, e.g., through a user device. The user can consider the action as a suggestion.

As another example, the system can process the output embedded representation 122 using a de-embedding machine learning model associated with an event type of test results. The system 100 thus generates the health-related event (“Labs”) 132b that has the event type of test result. In this example, the test result represents a predicted impact. The system can provide data representing the predicted impact for display to a user, e.g., through a user device. The user can thus use the system to simulate the impact of the health-related events 104a-n.

FIG. 2 shows predicted future health-related events 132a-b that have the event type of prescription and test results, but the predicted future health-related events can be of any event type.

FIG. 3 is a flow chart of an example process 300 for generating a predicted future health-related event associated with an individual. For convenience, the process 300 will be described as being performed by a system of one or more computers located in one or more locations. For example, a health-related event prediction system, e.g., the health-related event prediction system 100 depicted in FIG. 1, appropriately programmed in accordance with this specification, can perform the process 300.

The system identifies health-related data associated with the individual (step 302). The health-related data includes a sequence of health-related events.

In some implementations, the system can identify the health-related data from an electronic medical record for a corresponding patient. An electronic medical record is an electronic collection of health information for the corresponding patient. For example, the system can identify the electronic medical record for the patient from an electronic medical record repository and generate the sequence from the electronic medical record by identifying health events in the electronic medical record and ordering the health events by time. For example, the system can serialize the electronic medical record using interoperability standards such as the Fast Healthcare Interoperability Resources (FHIR) standard.

In some implementations, the system can append data identifying the time the health event occurred, e.g., a timestamp for the health-related event, or a relative time the health-related event occurred relative to other events in the sequence, to the data identifying the health event in the sequence.

In some implementations, the system can identify health-related data by receiving data representing one or more health-related events of the sequence from a user. The user can be a healthcare professional, for example.

The system can provide a user interface that allows a user to use the system. For example, the system can provide a user interface that receives inputs such as health-related data or proposed health-related events from the user. In some examples, the system can receive inputs that include natural language text. For example, the input can include a query in natural language about predicted future health-related events.

As another example, the input can include a natural language description of a proposed health-related event. The system can process the natural language description of the proposed health-related event to generate a health-related event that is described by the natural language description.

In some examples, one or more of the health-related events are recorded or actual health-related events, e.g., associated with individuals. In these examples, the recorded or actual health-related events can be de-identified. For example, the de-identified recorded or actual health-related events do not include identifying information about names, addresses, contact information, identifiers, etc., for the individuals or healthcare professionals. In some examples, the system processes recorded or actual health-related events to generate de-identified recorded or actual health-related events.

In some examples, one or more of the health-related events are proposed health-related events that are proposed by the user through a user interface of the system. For example, a healthcare professional can propose a health-related event that is a prescription and include the proposed health-related event in the health-related data. The system can process the health-related data to generate a prediction of the future health-related event. The prediction of the future health-related event can thus represent an impact of the proposed health-related event. The healthcare professional can thus use the system to simulate the impact of possible events on the patient's health.

The system generates, for each health-related event in the sequence of health-related events, a respective embedded representation of the health-related event (step 304). For example, each health-related event can be associated with an event type that is one of multiple event types. Each embedded representation can include one or more embeddings in a shared embedding space that is shared across the multiple event types. The system can process each health-related event using an embedding function corresponding to the event type of the health-related event to generate the respective embedded representation. Generating the respective embedded representation of a health-related event is described in further detail below with reference to FIG. 4.

The system generates, conditioned on at least the respective embedded representations of the health-related events, an output embedded representation of the future health-related event (step 306). For example, the system can generate the output embedded representation using a sequence processing neural network.

The sequence processing neural network can have any appropriate neural network architecture that allows the sequence processing neural network to process embedded representations of health-related events to generate an output embedded representation of one or more future health-related events. For example, the sequence processing neural network can include any appropriate types of neural network layers (e.g., embedding layers, fully connected layers, attention layers, and so forth) in any appropriate number (e.g., 2 layers, or 5 layers, or 10 layers) and connected in any appropriate configuration (e.g., as a directed graph of layers).

As particular examples, the sequence processing neural network can have a recurrent neural network (RNN) architecture, or a Transformer network architecture, e.g., encoder-only Transformer architectures, encoder-decoder Transformer architectures, decoder-only Transformer architectures, other attention-based architectures, and so on. Examples of such Transformer-based neural network architectures include those described in Gemini Team, et al. Gemini: a family of highly capable multimodal models. arXiv preprint arXiv: 2312.11805 (2023); and Ashish Vaswani, et al. “Attention is all you need,” Advances in neural information processing systems, 30, 2017.

In general a Transformer-based architecture can be one which is characterized by having a succession of self-attention neural network layers. A self-attention neural network layer has an attention layer input for each element of the input and is configured to apply an attention mechanism over attention layer inputs to generate an attention layer output for each element of the input. There are many different attention mechanisms that may be used.

As an example, a training system of the system or another training system can train the sequence processing neural network on a task that requires, given a current sequence of embedded representations, predicting the next embedded representation that follows the current sequence.

The sequence processing neural network can be pre-trained on a large dataset of training samples. A training system of the system or another training system can train the sequence processing neural network via optimization methods to reduce the error under a semi-supervised objective function. The sequence processing neural network can have been trained to minimize a loss function that measures a distance between output embedded representations of the future health-related event generated by the sequence processing neural network for training samples, and target output embedded representations for the training samples. The loss can be measured in the shared embedding space.

Each training sample can include (i) a training input that includes training embedded representations and (ii) a target output that includes a target output embedded representation, where the target output embedded representation is an embedded representation of the future health-related event for the training embedded representations. In some examples, the training samples are derived from recorded or actual health-related events associated with individuals. In these examples, the recorded or actual health-related events can be de-identified. For example, the de-identified recorded or actual health-related events do not include identifying information about names, addresses, contact information, identifiers, etc., for the individuals or healthcare professionals. In some examples, the system processes recorded or actual health-related events to generate de-identified recorded or actual health-related events.

For example, the training system can train the sequence processing neural network to minimize the discrepancy between the output embedded representation, that includes one or more n-dimensional vectors, produced by the sequence processing neural network for a health-related event of a training sample, and the target output embedded representation, that includes one or more n-dimensional vectors, of the training sample. The target output embedded representations and training embedded representations can include embedded representations for events that occurred in real data, or observations.

In some implementations, the training system can fine-tune the sequence processing neural network, for example, with human in the loop feedback. For example, after pre-training the sequence processing neural network, for one or more sequences of health-related events, the system can provide the predicted future health-related event for the sequence of health-related events for presentation to a user. In some examples, the one or more sequences are derived from recorded or actual health-related events associated with individuals. In these examples, the recorded or actual health-related events can be de-identified recorded or actual health-related events as described above.

The system can receive feedback from the user indicating the quality of the predicted future health-related event, e.g., that indicates the predicted future health-related event is likely, or that identifies a different future health-related event that is more likely. The system can generate a fine-tuning sample for the sequence of health-related events that includes a training input and a target output. The training input includes the sequence of health-related events. If the feedback indicates that the predicted future health-related event is likely, the target output can include the predicted future health-related event. If the feedback identifies a different future health-related event, the target output can include the different future health-related event. The system can further train, e.g., fine-tune, the sequence processing neural network on the fine-tuning samples.

In some implementations, the system can process a sequence of the embedded representations for the health-related events. The embedded representations can be ordered within the sequence according to a time at which the corresponding health-related event occurred.

For example, a sequence processing neural network that is a Transformer network can learn positional encodings that characterize the time at which each health-related event occurred. When applying the self-attention mechanism, the sequence processing neural network can modify the attention scores based on the positional encodings.

The system can also obtain a likelihood score for predicted health-related events and other computational byproducts. Examples of computational byproducts are described further below.

The system processes at least the output embedded representation of the future health-related event to generate the prediction of the future health-related event (step 308). For example, the system can process at least the output embedded representation using one or more de-embedding machine learning models.

For example, the one or more de-embedding machine learning models can include classifiers, probabilistic models, neural networks, language models, etc.

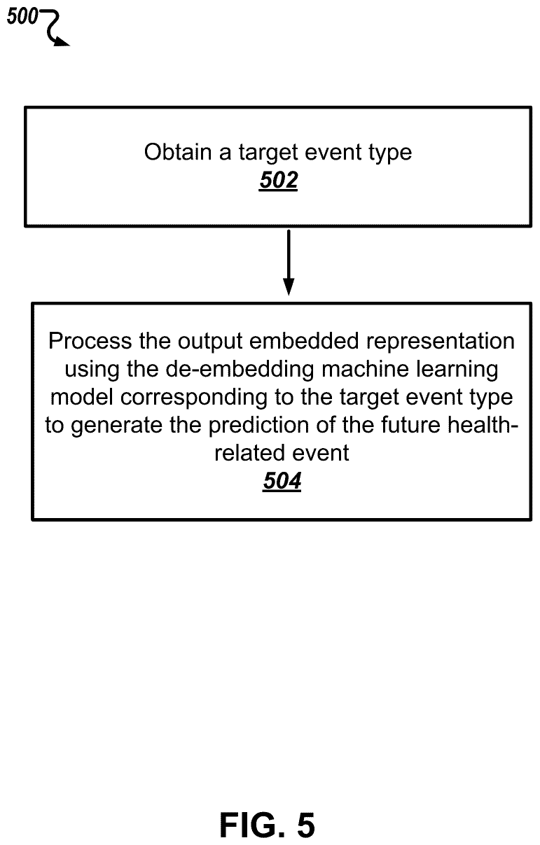

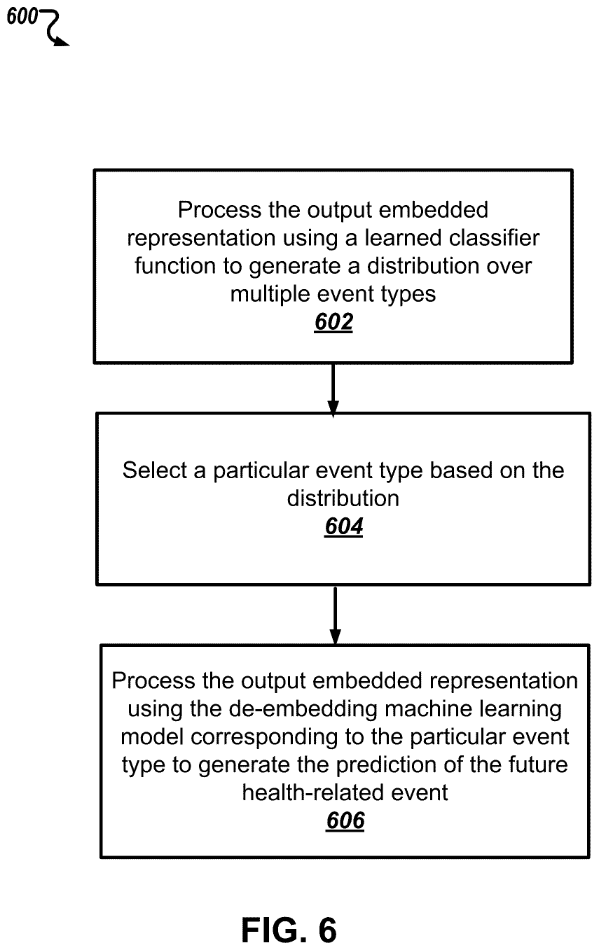

In some implementations, each of the one or more de-embedding machine learning models can correspond to a particular event type. In some examples, the system can generate the prediction of the future health-related event using the de-embedding machine learning model corresponding to a target event type, as described below with reference to FIG. 5. In other examples, the system can generate the prediction of the future health-related event using the de-embedding machine learning model corresponding to a particular event type determined by the system, as described below with reference to FIG. 6.

Each of the de-embedding machine learning models that correspond to a particular event type can be trained to map an embedded representation of a health-related event to a prediction of the health-related event of the particular event type.

When configured as a neural network, the de-embedding machine learning model can have any appropriate neural network architecture that allows the de-embedding machine learning model to map one or more embeddings in the shared embedding space to a health-related event associated with a particular event type. For example, the de-embedding machine learning model can include any appropriate types of neural network layers (e.g., embedding layers, fully connected layers, attention layers, and so forth) in any appropriate number (e.g., 2 layers, or 5 layers, or 10 layers) and connected in any appropriate configuration (e.g., as a directed graph of layers).

In some examples, the de-embedding machine learning model can include the decoder of an autoencoder neural network corresponding to the respective event type that also includes an encoder. The system can train the autoencoder neural network to optimize an objective function that measures an error (e.g., a reconstruction error) between (i) a health-related event that has been generated using the encoder and the decoder for a training health-related event and (ii) the training health-related event. In some examples, the training health-related event is a recorded or actual health-related event associated with an individual. In these examples, the recorded or actual health-related event can be a de-identified recorded or actual health-related event as described above. The system can determine gradients of the objective function with respect to current values of parameters of the autoencoder neural network, e.g., using backpropagation. The system can then update the current values of the parameters of the autoencoder neural network using the gradients, e.g., by the update rule of an appropriate gradient descent optimization algorithm, e.g., RMSprop or Adam.

In some implementations, the one or more de-embedding machine learning models can include a generative neural network. The system can process the output embedded representation using the generative neural network to generate the prediction of the future health-related event. The generative neural network can have been trained to generate a prediction of a future health-related event conditioned on the output embedded representation. For example, the generative neural network can have been trained on training samples that each include a training input that includes an embedded representation and a target output that includes the health-related event represented by the embedded representation. In some examples, the generative neural network can have been trained to generate predictions of future health-related events associated with different event types. In some examples, the generative neural network can include a text generation neural network, an image generation neural network, and/or an audio generation neural network.

In some examples, the training samples are derived from recorded or actual health-related events associated with individuals. In these examples, the recorded or actual health-related events can be de-identified. For example, the de-identified recorded or actual health-related events do not include identifying information about names, addresses, contact information, identifiers, etc., for the individuals or healthcare professionals. In some examples, the system processes recorded or actual health-related events to generate de-identified recorded or actual health-related events.

The prediction of the health-related event can include any of the event types described above. For example, the health-related event can be a predicted lab result. As another example, the health-related event can be an action such as a prescription.

In some examples, the future health-related event can be an action, e.g., a procedure or prescription. The system can provide data representing the future health-related event to the user. The user can consider the action as a suggestion.

In some implementations, the system can evaluate the predicted action for the patient. For example, the system can include the action in the sequence of health-related events, and process the new sequence of health-related events to predict a future health-related event. The future health-related event can be, e.g., a predicted lab result, and represent the predicted impact of taking the action.

In some implementations, the system can provide a score for the predicted action for the patient. For example, the system can determine a score for the action based on a likelihood and/or the impact of taking the action. The system can determine the likelihood for a predicted health-event based on the logits (e.g., scores or inputs to a soft-max layer) output by the sequence processing neural network. As an example, the system can determine a higher score for an action with a higher likelihood, and a higher score for an action with a more positive predicted impact of taking the action.

In some implementations, the system can compute the relative or absolute likelihoods of the future health-related event. For example, the system can compute the likelihood of a future health-related event given a proposed action or counterfactual circumstance.

In some implementations, the system can compute the relative or absolute likelihoods of proposed future health-related events. For example, the system can identify data representing a proposed future health-related event, e.g., that is received from a user. The system can generate an embedded representation of the proposed future health-related event, e.g., using the embedding function corresponding to the event type of the proposed future health-related event. The system can process health-related data as described above to generate a predicted future health-related event. The system can process a distribution over health-related events, e.g., output by the sequence processing neural network during processing of the health-related data, to determine a likelihood for the embedded representation of the proposed future health-related event. In some examples, the system can determine the likely time course of an event. For example, the system can compute statistics such as the proportion of multiple sequences of predicted health-related events that include the proposed future health-related event at particular times.

In some implementations, the system can provide prompts to a user about the proposed future health-related event. For example, the proposed future health-related event may have a likelihood that is below a threshold likelihood. The system can determine that the proposed health-related event is a commission event, e.g., an event that is predicted to have a low probability, or a suboptimal decision, e.g., an event that breaks with routine practice. Conventional systems for monitoring health-related events may not accurately detect commission events or suboptimal decisions.

For example, the system can determine that a proposed action of ordering an antibiotic related to a medicine for a patient that is allergic to the medicine has a low likelihood, and can provide a prompt to the user about the proposed action. As a particular example, the health-related data can include that the patient has a serious allergy to penicillin. The proposed future health-related event can include a prescription for a related antibiotic, nafcillin. A potential result of giving the patient the related antibiotic can include, e.g., the patient going into shock and not being able to breathe, getting intubated and going on life support, staying in the hospital for an extra period of time, and being discharged to a care facility instead of home. The system can determine whether the likelihood of the proposed health-related event occurring is unlikely, e.g., below the threshold likelihood, by processing the health-related data as described above. In response to determining that the nafcillin prescription is unlikely, the system can provide a prompt to the user.

As another example, the system can determine that a proposed action of ordering a procedure for a condition for a patient whose medical history does not include the condition has a low likelihood, and can provide a prompt to the user about the proposed action. As a particular example, Patient A may have kidney failure, while Patient B may be in a coma. A healthcare professional may accidentally order an invasive dialysis catheter for Patient B, that is meant for Patient A. The system can determine whether the likelihood of the proposed health-related event occurring is unlikely, e.g., below the threshold likelihood, by processing the health-related data for Patient B as described above. In response to determining that the dialysis catheter procedure is unlikely, the system can provide a prompt to the user.

As another example, the system can determine that a proposed action of starting chemotherapy for a patient whose medical history does not include cancer has a low likelihood, and can provide a prompt to the user about the proposed action. As a particular example, a healthcare professional intending to order lomotil (an anti-diarrhea medication) may order lomustine instead (a chemotherapy for cancer). The system can determine whether the likelihood of the proposed health-related event occurring is unlikely, e.g., below the threshold likelihood, by processing the health-related data as described above. In response to determining that the prescription for lomustine is unlikely, the system can provide a prompt to the user.

In some implementations, the system can provide prompts to the user that include information about a predicted future health-related event that has a higher likelihood than the proposed action, for example, as a suggested action. For example, the system can provide data representing one or more predicted health-related events generated using the system. In some implementations, the prompts can also include information about the potential impacts of the proposed actions, e.g., identified by generating a predicted future health-related event for a sequence of health-related events that includes the proposed actions.

For example, the system can determine that a proposed action of prescribing a certain medication is a suboptimal decision, and can provide a prompt to the user about the proposed action, a suggested action, or both. As a particular example, a healthcare professional may prescribe ceftriaxone for a dialysis patient that has sepsis and blood cultures return with “gram positive cocci.” A more routine choice would be to prescribe vancomycin because dialysis patients with these kinds of infections often have a bacteria that is resistant to ceftriaxone. The system can process health-related data for the dialysis patient as described above, and determine that the action of prescribing ceftriaxone is unlikely. The system can generate one or more predicted future health-related events that indicate the action of prescribing vancomycin is more likely.

FIG. 4 is a flow chart of an example process 400 for generating an embedded representation of a health-related event. For convenience, the process 400 will be described as being performed by a system of one or more computers located in one or more locations. For example, a health-related event prediction system, e.g., the health-related event prediction system 100 depicted in FIG. 1, appropriately programmed in accordance with this specification, can perform the process 400.

The system can perform the process 400 for each of the health-related events in the sequence of health-related events of the health-related data identified in step 302 of FIG. 3 above.

The system determines a respective event type for the health-related event (step 402). As an example, the system can process the health-related event to determine the respective event type. For example, the system can extract the event type from the FHIR resource for the health-related event. As another example, the system can process the health-related event using a learned classifier function that generates a distribution over the multiple event types for the health-related event. The system can determine the respective event type as the event type with the highest probability in the distribution.

The system processes the health-related event using an embedding function corresponding to the respective event type to generate the respective embedded representation of the health-related event (step 404). The embedding function can include a learned function.

In some examples, the embedding function can include any data values that define a learned mapping to generate one or more embeddings in the shared embedding space from a health-related event provided as input to the embedding function.

In some examples, the embedding function for one or more of the event types includes an embedding neural network.

The embedding neural network can have any appropriate neural network architecture that allows the embedding neural network to map a health-related event associated with a particular event type to one or more embeddings in the shared embedding space. For example, the embedding neural network can include any appropriate types of neural network layers (e.g., embedding layers, fully connected layers, attention layers, and so forth) in any appropriate number (e.g., 2 layers, or 5 layers, or 10 layers) and connected in any appropriate configuration (e.g., as a directed graph of layers).

As an example, the embedding neural network can include an image embedding neural network, e.g., can have a vision Transformer (ViT) architecture that includes one or more attention layers. As another example, the embedding neural network can have a convolutional neural network architecture that includes one or more convolutional layers. Other examples of embedding neural networks include text embedding neural networks, audio embedding neural networks, or video embedding neural networks.

In some examples, the embedding neural network can be the encoder of an autoencoder neural network corresponding to the respective event type that also includes a decoder. Training the autoencoder neural network is described below in further detail with reference to FIG. 6.

In some examples, an embedding neural network can be pre-trained on unlabeled training data based on optimizing a self-supervised or unsupervised loss function to generate embeddings in the shared embedding space that have a fixed dimensionality.

In some examples, the training data are derived from recorded or actual health-related events associated with individuals. In these examples, the recorded or actual health-related events can be de-identified. For example, the de-identified recorded or actual health-related events do not include identifying information about names, addresses, contact information, identifiers, etc., for the individuals or healthcare professionals. In some examples, the system processes recorded or actual health-related events to generate de-identified recorded or actual health-related events.

In some implementations, the embedding neural network can be pre-trained and frozen prior to training the sequence-processing neural network discussed above. For example, during training of the sequence processing neural network, the system does not adjust the pre-trained parameter values of the embedding neural network.

In other implementations, the embedding neural network can be pre-trained, and parameters for the embedding neural network can be updated during training of the sequence processing neural network. For example, the embedding neural network can be optimized along with the sequence processing neural network to reduce the total error on training data through back propagation or the chain rule.

The embedding neural network can also be fine-tuned, for example, through human in the loop feedback. For example, the system can provide data representing a set of health-related events for the particular event type for presentation to a user. The system can receive feedback from the user that indicates that a subset of the health-related events are more similar. The system can update the parameters of the embedding neural network to generate embeddings for the subset of the health-related events that are closer in the embedding space.

In some implementations, the system can obtain a corresponding event type encoding for each event type that characterizes the event type. For example, the system can maintain a mapping of event types and corresponding event type encodings. The system can update each embedded representation with the corresponding event type encoding. For example, each event type encoding can be a vector, and the system can append or prepend the vector to each embedded representation.

In some implementations, the system can update each embedded representation by applying a corresponding learned transformation for the event type to the embedded representation. By applying the corresponding learned transformation for the event type, the system can adjust the embedded representation to be closer in the embedding space to other embedded representations of the event type. The learned transformation can be learned, for example, based on a loss function that measures a distance between embedded representations for health-related events of the event type and other embedded representations for health-related events of other event types. The learned transformation can be, for example, a linear transformation applied using a feedforward layer, or a nonlinear transformation applied using a multilayer perceptron (MLP).

In some implementations, each embedded representation can represent a time at which the health-related event occurred. For example, each embedded representation can represent an absolute time, e.g., a timestamp, for the health-related event. In some examples, each embedded representation can include a positional encoding representing the position of the embedded representation within the sequence of embedded representations.

FIG. 5 is a flowchart of an example process 500 for processing an output embedded representation using a de-embedding machine learning model. For convenience, the process 500 will be described as being performed by a system of one or more computers located in one or more locations. For example, a health-related event prediction system, e.g., the health-related event prediction system 100 depicted in FIG. 1, appropriately programmed in accordance with this specification, can perform the process 500.

The system can obtain a target event type (step 502). For example, the system can obtain the target event type from a user. For example, the system can provide data representing each of the event types for presentation to the user. The system can receive an input from the user that identifies a selected event type of the event types. As an example, as shown in FIG. 2, the target event type can be a prescription.

The system can process the output embedded representation using the de-embedding machine learning model that corresponds to the target event type to generate the prediction of the future health-related event (step 504). For example, as described above with reference to FIG. 3, each of the de-embedding machine learning models corresponds to a respective event type. By processing the output embedded representation using the de-embedding machine learning model that corresponds to the target event type, the system can generate a future health-related event of the target event type.

FIG. 6 is a flowchart of another example process 600 for processing an output embedded representation using a de-embedding machine learning model. For convenience, the process 600 will be described as being performed by a system of one or more computers located in one or more locations. For example, a health-related event prediction system, e.g., the health-related event prediction system 100 depicted in FIG. 1, appropriately programmed in accordance with this specification, can perform the process 600.

The system processes the output embedded representation using a learned classifier function to generate a distribution over multiple event types (step 602). The distribution includes a probability for each of the multiple event types.

For example, the learned classifier function can include a machine learning model such as a decision-tree based model or a neural network. When configured as a neural network, the learned classifier function can include any appropriate types of neural network layers (e.g., convolutional layer, fully connected layers, attention layers, pooling layers, and so forth) in any appropriate number (e.g., 1 layer, or 5 layers, or 10 layers) and connected in any appropriate configuration (e.g., as a directed graph of layers).

The learned classifier function can be trained on a set of training samples by a machine learning technique. Each of the training samples can include (i) a training input that includes an embedded representation, and (ii) a target event type for the embedded representation.

For each training sample, the system can evaluate an objective function that measures an error (e.g., a cross-entropy loss) for each training sample between (i) a distribution over event types generated by the learned classifier function by processing the training input of the training sample, and (ii) the target event type of the training sample. The system can determine gradients of the objective function with respect to current values of parameters of the learned classifier function, e.g., using backpropagation. The system can then update the current values of the parameters of the learned classifier function using the gradients, e.g., by the update rule of an appropriate gradient descent optimization algorithm, e.g., RMSprop or Adam.

The system can select one of the event types based on the distribution (step 604). For example, the system can select the event type with a highest probability.

The system can process the output embedded representation using the de-embedding machine learning model that corresponds to the selected event type to generate the prediction of the future health-related event (step 606). For example, as described above with reference to FIG. 3, each of the de-embedding machine learning models corresponds to a respective event type. By processing the output embedded representation using the de-embedding machine learning model that corresponds to the selected event type, the system can generate a future health-related event of the selected event type.

FIG. 7 is a flowchart of an example process 700 for obtaining a prediction of a future health-related event associated with an individual. For convenience, the process 700 will be described as being performed by a system of one or more computers located in one or more locations. For example, a health-related event prediction system, e.g., the health-related event prediction system 100 depicted in FIG. 1, appropriately programmed in accordance with this specification, can perform the process 700. In particular, the process 700 can be performed by a health-related event prediction system that includes embedding functions.

The system identifies health-related data associated with an individual (step 702). For example, the system can identify health-related data as described with reference to step 302 of FIG. 3.

The system generates, for each health-related event in the sequence of health-related events, a respective embedded representation of the health-related event (step 704). For example, the system can generate respective embedded representations as described with reference to step 304 of FIG. 3.

The system obtains a prediction of a future health-related event associated with the individual (step 706). For example, the system provides the respective embedded representations to one or more machine learning models of a separate system. The one or more machine learning models can include, for example, a sequence processing neural network and one or more de-embedding machine learning models. The system can receive the prediction of the future health-related event from the separate system.

By providing embedded representations of health-related events to another system, the system can maintain data security and data privacy of the health-related events while reducing the computational resources that would otherwise be required to store and perform inference using the sequence processing neural network and the one or more de-embedding machine learning models.

FIG. 8 is a flowchart of an example process 800 for obtaining a prediction of a future health-related event associated with an individual. For convenience, the process 800 will be described as being performed by a system of one or more computers located in one or more locations. For example, a health-related event prediction system, e.g., the health-related event prediction system 100 depicted in FIG. 1, appropriately programmed in accordance with this specification, can perform the process 800. In particular, the process 800 can be performed by a health-related event prediction system that includes a sequence processing neural network.

The system receives a sequence of embedded representations of health-related events associated with an individual (step 802). For example, the system can receive the sequence of embedded representations from a separate system. As an example, the separate system can include one or more embedding functions.

The system generates, conditioned on at least the sequence of embedded representations, an output embedded representation of a future health-related event associated with the individual (step 804). For example, the system can generate the output embedded representation using a sequence processing neural network as described with reference to step 306 of FIG. 3.

The system obtains a prediction of a future health-related event associated with the individual (step 806). For example, the system provides the output embedded representation to one or more machine learning models of a separate system. The system can receive the prediction of the future health-related event from the separate system.

By receiving embedded representations of health-related events from another system, the system can maintain data security and data privacy of the health-related events. The system can also maintain data security and data privacy of the future health-related event by providing the output embedded representation of the future health-related event to a separate system, while reducing the computational resources that would otherwise be required to store and perform inference using the embedding functions and the one or more de-embedding machine learning models.

FIG. 9 is a flowchart of an example process 900 for generating a prediction of a future health-related event associated with an individual. For convenience, the process 900 will be described as being performed by a system of one or more computers located in one or more locations. For example, a health-related event prediction system, e.g., the health-related event prediction system 100 depicted in FIG. 1, appropriately programmed in accordance with this specification, can perform the process 900. In particular, the process 700 can be performed by a health-related event prediction system that includes one or more de-embedding machine learning models.

The system receives an output embedded representation of a future health-related event associated with an individual (step 902). For example, the system can receive the output embedded representation from one or more machine learning models of a separate system. The one or more machine learning models can include, for example, a sequence processing neural network. In some examples, the separate system can also include one or more embedding functions.

The system processes at least the output embedded representation to generate a prediction of the future health-related event associated with the individual (step 904). For example, the system can process at least the output embedded representation using one or more de-embedding machine learning models, as described above with reference to step 308 to FIG. 3.

By receiving an output embedded representation of a future health-related event from another system, the system can maintain data security and data privacy of the future health-related event, while reducing the computational resources that would otherwise be required to store and perform inference using the embedding functions and the sequence processing neural network.

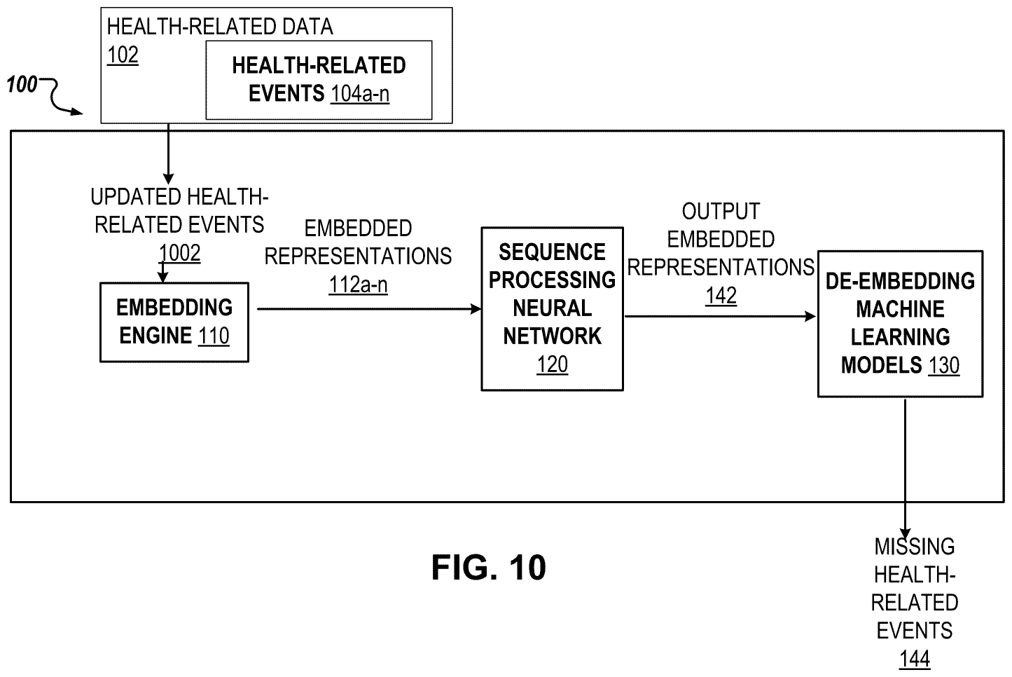

FIG. 10 shows the example health-related event prediction system 100 described above with reference to FIG. 1. In particular, in the example of FIG. 10, the health-related event prediction system 100 generates a prediction of one or more missing health-related events 144.

The system 100 identifies health-related data 102 as described above with reference to FIG. 1. In the example of FIG. 10, the health-related data is incomplete health-related data. For example, the sequence of health-related events 104a-n include one or more gaps to be filled. Each gap represents a missing health-related event that is likely to occur in the sequence in the context of the other health-related events 104a-n, but did not occur in the sequence of health-related events 104a-n. In some examples, the system 100 can identify the gaps, e.g., if the time interval between a preceding health-related event and a preceding health-related event meets a threshold time interval.

The system 100 updates the sequence of health-related events 104a-n by applying a mask for each of the one or more gaps. For example, the system 100 can replace each gap with a [MASK] token. The system 100 can thus generate a sequence of health-related events and one or more masks 1002.

The system 100 generates the embedded representations 112a-n as described with reference to FIG. 1 above. In the example of FIG. 10, the embedded representations 112a-n include an embedded representation of each health-related event and mask in the updated sequence of health-related events 1002.

The system 100 generates, for each mask in the updated sequence of health-related events 1002, an output embedded representation. For example, the system 100 can process the embedded representations 112a-n using the sequence processing neural network 120 to generate the output embedded representations 142. In the example of FIG. 10, the sequence processing neural network 120 is configured to generate a prediction of a health-related event 144 for each embedded representation of a mask in the embedded representations 112a-n based on the context of the embedded representations for health-related events in the embedded representations 112a-n.

The system 100 processes at least each of the output embedded representations 142 using the one or more de-embedding machine learning models 130, as described above with reference to FIG. 1, to generate the predictions of the missing health-related events 144.

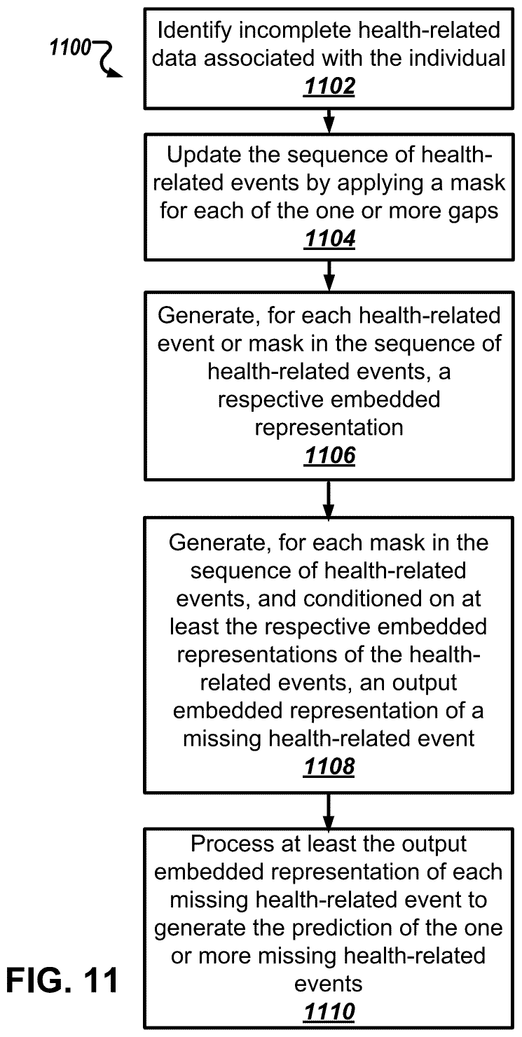

FIG. 11 is a flowchart of an example process 1100 for generating a prediction of a missing health-related event associated with an individual. For convenience, the process 1100 will be described as being performed by a system of one or more computers located in one or more locations. For example, a health-related event prediction system, e.g., the health-related event prediction system 100 depicted in FIG. 10, appropriately programmed in accordance with this specification, can perform the process 1100.

The system identifies incomplete health-related data associated with the individual (step 1102). The incomplete health-related data includes a sequence of health-related events with one or more gaps to be filled.

The system updates the sequence of health-related events by applying a mask for each of the one or more gaps (step 1104).

The system generates, for each health-related event or mask in the sequence of health-related events, a respective embedded representation of the health-related event or the mask (step 1106). For example, the system can use one or more embedding functions as described above to generate the respective embedded representation of each health-related event. In some examples, the system can train the embedding functions to generate an embedded representation for the mask.

The system generates, for each mask in the sequence of health-related events, and conditioned on at least the respective embedded representations of the health-related events, an output embedded representation of a missing health-related event (step 1108). For example, the system can generate each output embedded representation using a sequence processing neural network.

The sequence processing neural network can have any appropriate neural network architecture that allows the sequence processing neural network to process embedded representations of health-related events and masks to generate an output embedded representation of a health-related event for each mask. As a particular example, the sequence processing neural network can have an encoder-only Transformer architecture.

As an example, a training system of the system or another training system can train the sequence processing neural network on a task that requires, given a current sequence of embedded representations of health-related events and masks, the embedded representations that are likely to occur in place of the masks.

For example, the sequence processing neural network can be pre-trained on a large dataset of training samples. Each training sample can be derived from health-related data including a sequence of health-related events, and incomplete health-related data including the sequence of health-related events, where one or more health-related events have been replaced with a gap to be filled. Each training sample can include a training input that includes incomplete health-related data including the sequence of health-related events, and a training output that includes each health-related event that was replaced with a gap. For example, the one or more health-related events that are replaced with a gap to be filled can have been selected randomly.

In some examples, the training samples are derived from recorded or actual health-related events associated with individuals. In these examples, the recorded or actual health-related events can be de-identified. For example, the de-identified recorded or actual health-related events do not include identifying information about names, addresses, contact information, identifiers, etc., for the individuals or healthcare professionals. In some examples, the system processes recorded or actual health-related events to generate de-identified recorded or actual health-related events.

For each training sample, the system can evaluate an objective function that measures an error (e.g., a cross-entropy loss) for each training sample between (i) a distribution generated by the sequence processing neural network by processing the training input of the training sample, and (ii) the target output of the training sample. The system can determine gradients of the objective function with respect to current values of parameters of the sequence processing neural network, e.g., using backpropagation. The system can then update the current values of the parameters of the sequence processing neural network using the gradients, e.g., by the update rule of an appropriate gradient descent optimization algorithm, e.g., RMSprop or Adam.

In some implementations, the system can fine-tune the sequence processing neural network, for example, with human in the loop feedback. For example, after pre-training the sequence processing neural network, for one or more sequences of health-related events with gaps, the system can provide the predicted missing health-related events for the sequence of health-related events for presentation to a user. In some examples, the one or more sequences are derived from recorded or actual health-related events associated with individuals. In these examples, the recorded or actual health-related events can be de-identified recorded or actual health-related events as described above.

The system can receive feedback from the user indicating the quality of the predicted missing health-related event, e.g., that indicates the predicted health-related event is likely, or that identifies a different predicted health-related event that is more likely. The system can generate a fine-tuning sample for the sequence of health-related events that includes a training input and a target output. The training input includes the sequence of health-related events with gaps. If the feedback indicates that the predicted missing health-related event is likely, the target output can include the predicted missing health-related event. If the feedback identifies a different missing health-related event, the target output can include the different missing health-related event. The system can further train, e.g., fine-tune, the sequence processing neural network on the fine-tuning samples.

The system processes at least the output embedded representation of each missing health-related event to generate the prediction of the one or more missing health-related events (step 1110). For example, the system can process the output embedded representations using one or more de-embedding machine learning models as described above.

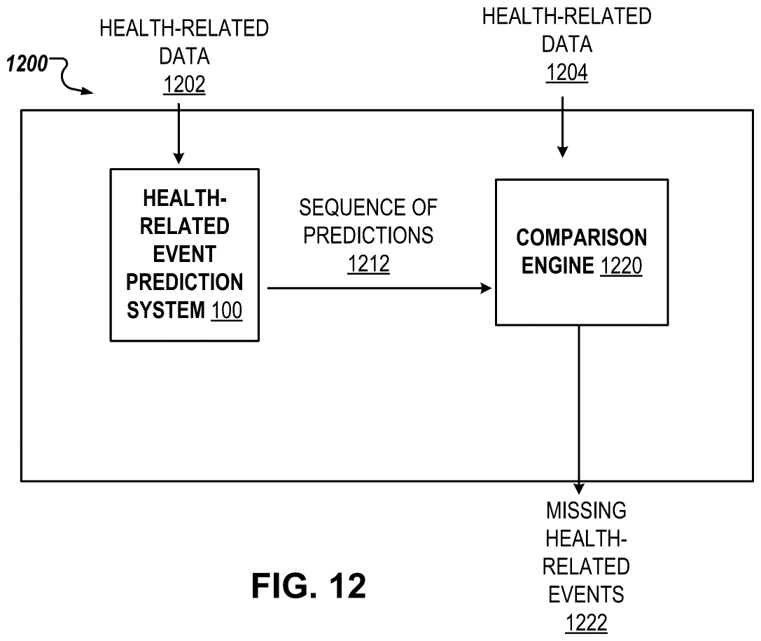

FIG. 12 shows an example health-related event detection system 1200. The health-related event detection system 1200 is an example of a system implemented as computer programs on one or more computers in one or more locations in which the systems, components, and techniques described below are implemented.

The health-related event detection system 1200 is configured to process health-related data 1202 associated with an individual at a first time, and health-related data 1204 associated with the individual at a second time, to identify one or more missing health-related events 1222. A missing health-related event is a health-related event that was likely to have occurred, but did not. For example, each missing health-related event can be a lab test that is commonly ordered given the health-related data 1202 for a patient, or a medication that is commonly prescribed given the health-related data 1202 for a patient. Conventional systems for monitoring health-related events may not accurately detect omissions.

The system 1200 can identify health-related data 1202, for example, as described above with identifying health-related data 102 of FIG. 1. The health-related data 1202 is associated with the individual at a first time.

In the example of FIG. 12, the system 1200 can identify health-related data 1202 as of Jan. 1, 2024. As an example, the health-related data 1202 can include a health-related event of initiating warfarin, a health-related event of a blood culture that shows that bacteria is resistant to ceftriaxone, and a health-related event of taking ceftriaxone.

The system 1200 can generate a sequence of predictions 1212 of future health-related events at a first point in time. For example, the sequence of predictions 1212 of future health-related events can include one or more health-related events that have a high likelihood of occurring in a window of time after the first point in time. For example, the system 1200 can generate the sequence of predictions 1212 using the health-related event prediction system 100 of FIG. 1. In some examples, the system 1200 can generate predictions using the health-related event prediction system 100 until data identifying the time of the predicted health-related events indicates that the time is after the window of time.

In the example of FIG. 12, the system 1200 can generate ten predictions of health-related events in the sequence of predictions 1212 that are conditioned on the health-related data 1202. The ten health-related events have a very high likelihood of occurring over the next week after Jan. 1, 2024. For example, one of the health-related events can include an order for a PT/INR, which is likely after initiating warfarin. Another example of a health-related event can include changing the antibiotic the patient is taking, which is likely since the blood culture showed that the bacteria is resistant to ceftriaxone, and the patient is on ceftriaxone.

The system 1200 can identify health-related data 1204, for example, as described above with identifying health-related data 102 of FIG. 1. The health-related data 1204 is associated with the individual at a second time. The second time is within or after the window of time. The health-related data 1204 can include one or more health-related events that occurred between the first time and the second time, e.g., that are not included in the health-related data 1202.

In the example of FIG. 12, the health-related data 1204 can include eight health-related events. The eight health-related events can have occurred during the week after Jan. 1, 2024.

The system can compare the sequence of predictions 1212 of future health-related events for the window of time with the health-related data 1204 for the window of time. For example, the system 1200 can use a comparison engine 1220 to compare the sequence of predictions 1212 with the health-related data 1204. The comparison engine 1220 can identify predictions of future health-related events within the sequence of predictions 1212 that do not have matches within the health-related data 1204 as missing health-related events 1222. Thus, the system 1200 can determine that some of the predictions of future health-related events did not occur and are likely to be missing health-related events.

In the example of FIG. 1, the system 1200 can determine that eight of the health-related events of the sequence of predictions 1212 occurred in the health-related data 1204. Thus the system 1200 identifies the two remaining health-related events of the sequence of predictions 1212 as missing health-related events 1222.

In some implementations, the system 1200 can provide prompts to a user based on the detection of missing health-related events 1222. As a particular example, a patient may have low blood potassium levels. A healthcare professional may order a potassium replacement, but the order does not go through. The patient may develop problems caused by the low potassium after a certain period of time, e.g., four hours. The system 1200 can determine that a potassium medication order has a high probability for being a future health-related event within a certain period of time, but there is no potassium medication order in the updated health-related data 1204. After a threshold period of time, e.g., that is shorter than the certain period of time, the system can provide a prompt to the user with a suggested potassium medication order.

In some implementations, the system 1200 can provide the predictions of missing health-related events 1222, or data derived from the predictions of missing health-related events 1222, for presentation to a user through a user interface on a user device.

In some implementations, components of the system 1200 such as the health-related event prediction system 100 can execute on one or more computers remote from the system 1200. In some implementations, the components can communicate with each other over a network. In some implementations, the components can communicate using one or more application programming interfaces (APIs). For example, the system 1200 can use an API for the health-related event prediction system 100 to provide inputs and receive outputs to the health-related event prediction system 100.

In some implementations, as described above, the embedding functions, sequence processing neural network, and/or de-embedding machine learning models of the health-related event prediction system 100 can execute separately from a user device. For example, in some implementations, the embedding functions can execute on the user device, and the sequence processing neural network and de-embedding machine learning models can run on one or more computers independently from the user device.

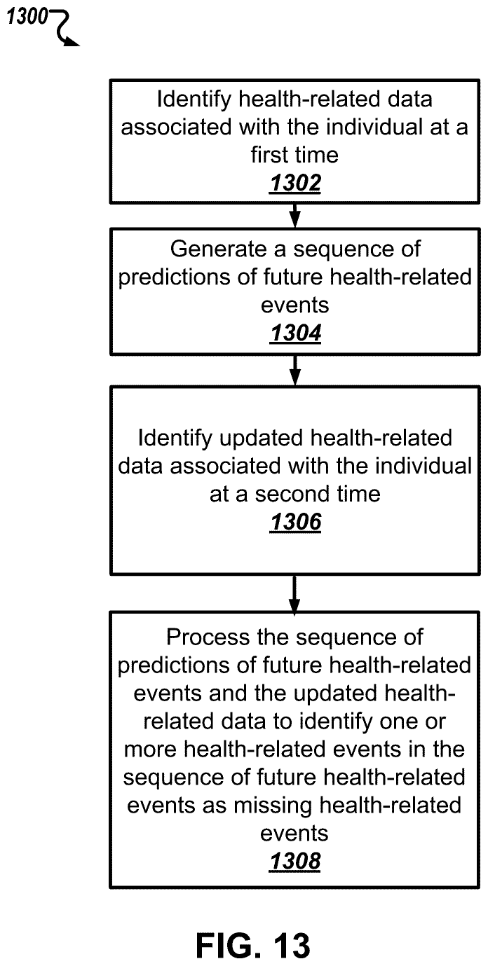

FIG. 13 is a flowchart of an example process 1300 identifying one or more missing health-related events associated with an individual. For convenience, the process 1300 will be described as being performed by a system of one or more computers located in one or more locations. For example, a health-related event detection system, e.g., the health-related event detection system 1200 depicted in FIG. 12, appropriately programmed in accordance with this specification, can perform the process 1300.

The system identifies health-related data associated with the individual at a first time (step 1302). The health-related data includes a sequence of health-related events, such as the health-related events described above.

The system generates a sequence of predictions of future health-related events (step 1304). The future health-related events are likely to occur within a window of time after the first time. For example, the system can generate the sequence of predictions using one or more machine learning models, e.g., a sequence processing neural network, as described above.

The system identifies updated health-related data associated with the individual at a second time (step 1306). The updated health-related data includes a sequence of health-related events that have occurred between the first time and the second time. For example, the updated health-related data includes one or more actual health-related events associated with the individual.

The system processes the sequence of predictions of future health-related events and the updated health-related data to identify one or more health-related events in the sequence of future health-related events as missing health-related events (step 1308). The missing health-related events do not have a match within the updated health-related data. Thus, the system identifies predicted health-related events that do not have a corresponding match within the actual health-related events as missing health-related events.

Matching events can include, for example, events that occur within a threshold time period of each other or in the same relative order compared to other health-related events, and are associated with the same event type, or events that are similar. The system can determine whether events are similar using the embedding functions corresponding to each of the event types of the events. For example, the system can process each event using the embedding function corresponding to the event type for the event. The system can determine that events are similar if the distance between the embedded representations meets a threshold distance.

In some examples, one or more of the health-related events of the health-related data associated with the individual at a first time, and the updated health-related data associated with the individual at a second time, are recorded or actual health-related events, e.g., associated with the individual. In these examples, the recorded or actual health-related events can be de-identified recorded or actual health-related events as described above.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework or a JAX framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

In addition to the embodiments described above, the following embodiments are also innovative:

Embodiment 1 is a method for generating a prediction of a future health-related event associated with an individual, the method comprising: identifying health-related data associated with the individual, the health-related data comprising a sequence of health-related events; generating, for each health-related event in the sequence of health-related events, a respective embedded representation of the health-related event; generating, using a sequence processing neural network and conditioned on at least the respective embedded representations of the health-related events, an output embedded representation of the future health-related event; and processing at least the output embedded representation of the future health-related event using one or more de-embedding machine learning models to generate the prediction of the future health-related event.

Embodiment 2 is the method of embodiment 1, wherein the health-related data comprises time information for each health-related event in the sequence of health-related events.

Embodiment 3 is the method of any of embodiments 1-2, wherein generating an output embedded representation of the future health-related event comprises:
processing a sequence of the respective embedded representations for the health-related events, wherein the respective embedded representations for the health-related events are ordered within the sequence according to a time at which each health-related event occurred.

Embodiment 4 is the method of any of embodiments 1-3, wherein each health-related event is associated with an event type that is one of a plurality of event types, and wherein each respective embedded representation comprises one or more embeddings in a shared embedding space that is shared across the plurality of event types.

Embodiment 5 is the method of embodiment 4, wherein the respective embedded representations for two or more of the plurality of event types comprise a different number of embeddings in the shared embedding space.

Embodiment 6 is the method of any of embodiments 1-5, wherein generating, for each health-related event in the sequence of health-related events, a respective embedded representation of the health-related event comprises: for each health-related event in the sequence of health-related events: determining a respective event type for the health-related event; and processing the health-related event using an embedding function corresponding to the respective event type to generate the respective embedded representation of the health-related event.

Embodiment 7 is the method of embodiment 6, wherein for one or more of the respective event types, the corresponding embedding function is a learned function.

Embodiment 8 is the method of embodiment 7, wherein the learned function is an embedding neural network.

Embodiment 9 is the method of embodiment 8, wherein the embedding neural network is pre-trained and frozen prior to training the sequence processing neural network.

Embodiment 10 is the method of embodiment 8, wherein the embedding neural network is pre-trained, and wherein parameters for the embedding neural network are updated during training of the sequence processing neural network.

Embodiment 11 is the method of any of embodiments 6-10, further comprising, for each health-related event in the sequence of health-related events: obtaining a corresponding event type encoding for the respective event type characterizing the respective event type; and updating the respective embedded representation using the corresponding event type encoding.

Embodiment 12 is the method of any of embodiments 6-10, further comprising, for each health-related event in the sequence of health-related events: updating the respective embedded representation by applying a corresponding learned transformation for the respective event type to the respective embedded representation.

Embodiment 13 is the method of embodiment 12, wherein the corresponding learned transformation for the respective event type is learned based on a loss function that measures a distance between embedded representations for health-related events of the respective event type and other embedded representations for health-related events of other event types.

Embodiment 14 is the method of any of embodiments 1-13, wherein the sequence processing neural network has been trained to minimize a loss function that measures a distance between output embedded representations of the future health-related event generated by the sequence processing neural network for training samples, and target output embedded representations for the training samples.

Embodiment 15 is the method of any of embodiments 1-14, wherein the sequence processing neural network comprises a Transformer network.

Embodiment 16 is the method of any of embodiments 1-14, wherein the sequence processing neural network comprises a recurrent neural network.

Embodiment 17 is the method of any of embodiments 1-16, wherein each of the one or more de-embedding machine learning models corresponds to a respective event type, and wherein processing at least the output embedded representation of the future health-related event using one or more de-embedding machine learning models to generate the prediction of the future health-related event comprises: obtaining a target event type; and processing the output embedded representation of the future health-related event using the de-embedding machine learning model corresponding to the target event type to generate the prediction of the future health-related event.

Embodiment 18 is the method of embodiment 17, wherein each of the one or more de-embedding machine learning models corresponding to a respective event type has been trained to map an embedded representation of a health-related event to a prediction of the health-related event, wherein the future health-related event is of the respective event type.

Embodiment 19 is the method of any of embodiments 1-16, wherein each of the one or more de-embedding machine learning models corresponds to a respective event type, and wherein processing at least the output embedded representation of the future health-related event using one or more de-embedding machine learning models to generate the prediction of the future health-related event comprises: processing the output embedded representation using a learned classifier function to generate a distribution over a plurality of event types; selecting a particular event type based on the distribution; and processing the output embedded representation using the de-embedding machine learning models corresponding to the particular event type to generate the prediction of the future health-related event.

Embodiment 20 is the method of any of embodiments 1-16, wherein the one or more de-embedding machine learning models comprise a generative neural network, and wherein processing at least the output embedded representation of the future health-related event using one or more de-embedding machine learning models to generate the prediction of the future health-related event comprises: processing the output embedded representation of the future health-related event using the generative neural network to generate the prediction of the future health-related event, wherein the generative neural network has been trained to generate a prediction of a future health-related event conditioned on the output embedded representation of the future health-related event.

Embodiment 21 is the method of any of embodiments 1-20, wherein identifying health-related data associated with the individual comprises receiving data representing one or more health-related events of the sequence from a user.

Embodiment 22 is the method of embodiment 21, wherein the one or more health-related events of the sequence comprise one or more recorded health-related events associated with the individual and one or more proposed health-related events.

Embodiment 23 is the method of embodiment 22, wherein the prediction of the future health-related event represents an impact of the one or more proposed health-related events.

Embodiment 24 is the method of any of embodiments 1-23, wherein the future health-related event comprises an action.

Embodiment 25 is the method of embodiment 24, wherein the method further comprises evaluating the action for the individual.

Embodiment 26 is a method for generating a prediction of one or more missing health-related events associated with an individual, the method comprising: identifying incomplete health-related data associated with the individual, the incomplete health-related data comprising a sequence of health-related events with one or more gaps to be filled; updating the sequence of health-related events by applying a mask for each of the one or more gaps; generating, for each health-related event or mask in the sequence of health-related events, a respective embedded representation of the health-related event or the mask; generating, for each mask in the sequence of health-related events, using a sequence processing neural network and conditioned on at least the respective embedded representations of the health-related events, an output embedded representation of a missing health-related event; and processing at least the output embedded representation of each missing health-related event using one or more de-embedding machine learning models to generate the prediction of the one or more missing health-related events.

Embodiment 27 is the method of embodiment 26, wherein the sequence processing neural network has been trained on training samples that are each derived from health-related data comprising a sequence of health-related events, and incomplete health-related data comprising the sequence of health-related events, wherein one or more health-related events have been replaced with a gap to be filled.

Embodiment 28 is a method for detecting one or more missing health-related events associated with an individual, the method comprising: identifying health-related data associated with the individual at a first time, the health-related data comprising a sequence of health-related events; generating a sequence of predictions of future health-related events that are likely to occur within a window of time after the first time; identifying updated health-related data associated with the individual at a second time, updated health-related data comprising a sequence of health-related events that have occurred between the first time and the second time; and processing the sequence of predictions of future health-related events and the updated health-related data to identify one or more health-related events in the sequence of future health-related events as missing health-related events, wherein the missing health-related events do not have a match within the updated health-related data.

Embodiment 29 is the method of embodiment 28, wherein generating a sequence of predictions of future health-related events that are likely to occur within a window of time after the first time comprises generating the sequence of predictions of future health-related events using a sequence processing neural network.

Embodiment 30 is a method comprising: identifying health-related data associated with an individual, the health-related data comprising a sequence of health-related events; generating, for each health-related event in the sequence of health-related events, a respective embedded representation of the health-related event; and obtaining a prediction of a future health-related event associated with the individual by providing the respective embedded representations to one or more machine learning models.

Embodiment 31 is a method comprising: receiving a sequence of embedded representations of health-related events associated with an individual; generating, using a sequence processing neural network and conditioned on at least the sequence of embedded representations, an output embedded representation of a future health-related event associated with the individual; and obtaining a prediction of a future health-related event associated with the individual by providing the output embedded representation to one or more machine learning models.

Embodiment 32 is a method comprising: receiving an output embedded representation of a future health-related event associated with an individual; and processing at least the output embedded representation using one or more de-embedding machine learning models to generate a prediction of the future health-related event associated with the individual.

Embodiment 33 is a system comprising one or more computers and one or more storage devices storing instructions that, when executed by the one or more computers, cause the one or more computers to perform the method of any one of embodiments 1-32.

Embodiment 34 is one or more computer-readable storage media storing instructions that when executed by one or more computers cause the one or more computers to perform the method of any of embodiments 1-32.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method for generating a prediction of a future health-related event associated with an individual, the method comprising:
identifying health-related data associated with the individual, the health-related data comprising a sequence of health-related events;
generating, for each health-related event in the sequence of health-related events, a respective embedded representation of the health-related event;
generating, using a sequence processing neural network and conditioned on at least the respective embedded representations of the health-related events, an output embedded representation of the future health-related event; and
processing at least the output embedded representation of the future health-related event using one or more de-embedding machine learning models to generate the prediction of the future health-related event.

2. The method of claim 1, wherein the health-related data comprises time information for each health-related event in the sequence of health-related events.

3. The method of claim 1, wherein generating an output embedded representation of the future health-related event comprises:
processing a sequence of the respective embedded representations for the health-related events, wherein the respective embedded representations for the health-related events are ordered within the sequence according to a time at which each health-related event occurred.

4. The method of claim 1, wherein each health-related event is associated with an event type that is one of a plurality of event types, and wherein each respective embedded representation comprises one or more embeddings in a shared embedding space that is shared across the plurality of event types.

5. The method of claim 4, wherein the respective embedded representations for two or more of the plurality of event types comprise a different number of embeddings in the shared embedding space.

6. The method of claim 1, wherein generating, for each health-related event in the sequence of health-related events, a respective embedded representation of the health-related event comprises:
for each health-related event in the sequence of health-related events:
determining a respective event type for the health-related event; and
processing the health-related event using an embedding function corresponding to the respective event type to generate the respective embedded representation of the health-related event.

7. The method of claim 6, wherein for one or more of the respective event types, the corresponding embedding function is a learned function.

8. The method of claim 7, wherein the learned function is an embedding neural network.

9. The method of claim 8, wherein the embedding neural network is pre-trained and frozen prior to training the sequence processing neural network.

10. The method of claim 8, wherein the embedding neural network is pre-trained, and wherein parameters for the embedding neural network are updated during training of the sequence processing neural network.

11. The method of claim 6, further comprising, for each health-related event in the sequence of health-related events:
obtaining a corresponding event type encoding for the respective event type characterizing the respective event type; and
updating the respective embedded representation using the corresponding event type encoding.

12. The method of claim 6, further comprising, for each health-related event in the sequence of health-related events:
updating the respective embedded representation by applying a corresponding learned transformation for the respective event type to the respective embedded representation.

13. The method of claim 1, wherein the sequence processing neural network has been trained to minimize a loss function that measures a distance between output embedded representations of the future health-related event generated by the sequence processing neural network for training samples, and target output embedded representations for the training samples.

14. The method of claim 1, wherein each of the one or more de-embedding machine learning models corresponds to a respective event type, and wherein processing at least the output embedded representation of the future health-related event using one or more de-embedding machine learning models to generate the prediction of the future health-related event comprises:
obtaining a target event type; and
processing the output embedded representation of the future health-related event using the de-embedding machine learning model corresponding to the target event type to generate the prediction of the future health-related event.

15. The method of claim 14, wherein each of the one or more de-embedding machine learning models corresponding to a respective event type has been trained to map an embedded representation of a health-related event to a prediction of the health-related event, wherein the future health-related event is of the respective event type.

16. The method of claim 1, wherein each of the one or more de-embedding machine learning models corresponds to a respective event type, and wherein processing at least the output embedded representation of the future health-related event using one or more de-embedding machine learning models to generate the prediction of the future health-related event comprises:
processing the output embedded representation using a learned classifier function to generate a distribution over a plurality of event types;
selecting a particular event type based on the distribution; and
processing the output embedded representation using the de-embedding machine learning model corresponding to the particular event type to generate the prediction of the future health-related event.

17. The method of claim 1, wherein the one or more de-embedding machine learning models comprise a generative neural network, and wherein processing at least the output embedded representation of the future health-related event using one or more de-embedding machine learning models to generate the prediction of the future health-related event comprises:
processing the output embedded representation of the future health-related event using the generative neural network to generate the prediction of the future health-related event, wherein the generative neural network has been trained to generate a prediction of a future health-related event conditioned on the output embedded representation of the future health-related event.

18. The method of claim 1, wherein identifying health-related data associated with the individual comprises receiving data representing one or more health-related events of the sequence from a user.

19. A method for generating a prediction of one or more missing health-related events associated with an individual, the method comprising:
identifying incomplete health-related data associated with the individual, the incomplete health-related data comprising a sequence of health-related events with one or more gaps to be filled;
updating the sequence of health-related events by applying a mask for each of the one or more gaps;
generating, for each health-related event or mask in the sequence of health-related events, a respective embedded representation of the health-related event or the mask;
generating, for each mask in the sequence of health-related events, using a sequence processing neural network and conditioned on at least the respective embedded representations of the health-related events, an output embedded representation of a missing health-related event; and
processing at least the output embedded representation of each missing health-related event using one or more de-embedding machine learning models to generate the prediction of the one or more missing health-related events.

20. A method for detecting one or more missing health-related events associated with an individual, the method comprising:
identifying health-related data associated with the individual at a first time, the health-related data comprising a sequence of health-related events;
generating a sequence of predictions of future health-related events that are likely to occur within a window of time after the first time;
identifying updated health-related data associated with the individual at a second time, updated health-related data comprising a sequence of health-related events that have occurred between the first time and the second time; and
processing the sequence of predictions of future health-related events and the updated health-related data to identify one or more health-related events in the sequence of future health-related events as missing health-related events, wherein the missing health-related events do not have a match within the updated health-related data.
